(19) United States
(12) Patent Application Publication
(10) Pub. No.: US 2021/0145806 A1
(43) Pub. Date: May 20, 2021

(54) Methods of Treating Brain Cancer and Related Diagnostic Methods

(71) Applicants: Emory University, Atlanta, GA (US); Children's Healthcare of Atlanta, Inc., Atlanta, GA (US)

(72) Inventors: Tobey MacDonald, Decatur, GA (US); Anna Marie Kenney, Atlanta, GA (US); Abhinav Dey, Decatur, GA (US); James Felker, Aspinwall, PA (US)

(21) Appl. No.: 16/952,887

(22) Filed: Nov. 19, 2020

Related U.S. Application Data
(60) Provisional application No. 62/938,252, filed on Nov. 20, 2019.

(51) Int. Cl. A61K 31/44; A61K 31/353; A61K 45/06; G01N 33/50; A61P 35/00

(52) U.S. Cl. CPC A61K 31/44; A61K 31/353; A61P 35/00; G01N 33/5011; A61K 45/06

(57) ABSTRACT

This disclosure relates to the treatment of brain cancer, e.g., medulloblastoma using a combination of STAT3 and YB-1 inhibitors. In certain embodiments, the STAT3 inhibitor is 3-(6-bromopyridin-2-yl)-2-cyano-N-(1-phenylethyl)acrylamide (WP1066) or salts thereof and YB-1 inhibitor is 2-(3,4-dihydroxyphenyl)-3,7-dihydroxy-4H-chromen-4-one (Fisetin) or salts thereof. In certain embodiments, this disclosure relates to methods of diagnosing and treating a subject diagnosed with medulloblastoma using assays disclosed herein.

### CROSS-REFERENCE TO RELATED APPLICATIONS

This application claims the benefit of U.S. Provisional Application No. 62/938,252 filed Nov. 20, 2019. The entirety of this application is hereby incorporated by reference for all purposes.

### BACKGROUND

Medulloblastoma (MB) are the most common solid pediatric malignant tumors. These tumors typically arise in young children from dividing progenitor cells in the cerebellum. Current treatments for medulloblastoma, surgery, craniospinal radiation, and chemotherapy, leave survivors with life-long, devastating side effects. Moreover, medulloblastoma recurrence and metastasis are lethal. Thus, there is a need to develop improved medulloblastoma therapies.

Dey et al. report YB-1 is elevated in medulloblastoma and drives proliferation in Sonic hedgehog dependent cerebellar granule neuron progenitor cells and medulloblastoma cells. Oncogene, 2016, 35(32): 4256-4268.

Felker et al. report organotypic tumor slice culture for evaluating treatment of medulloblastoma. Neuro-Oncology, 2016, 18(6), vi151. Abstracts from the 21st Annual Scientific Meeting and Education Day of the Society for Neuro-Oncology.

References cited herein are not an admission of prior art.

### SUMMARY

This disclosure relates to the treatment of brain cancer, e.g., medulloblastoma, using a combination of STAT3 and YB-1 inhibitors. In certain embodiments, the STAT3 inhibitor is 3-(6-bromopyridin-2-yl)-2-cyano-N-(1-phenylethyl)acrylamide (WP1066) or salts thereof and the YB-1 inhibitor is 2-(3,4-dihydroxyphenyl)-3,7-dihydroxy-4H-chromen-4-one (Fisetin) or salts thereof. In certain embodiments, this disclosure relates to methods of diagnosing and treating a subject diagnosed with medulloblastoma using assays disclosed herein.

In certain embodiments, this disclosure relates to methods of treating brain cancer comprising administering and effective amount of a STAT3 inhibitor in combination with a YB-1 inhibitor to a subject in need thereof. In certain embodiments, the subject is a human. In certain embodiments, the subject is diagnosed with medulloblastoma. In certain embodiments, the STAT3 inhibitor is 3-(6-bromopyridin-2-yl)-2-cyano-N-(1-phenylethyl)acrylamide (WP1066) or salts thereof and the YB-1 inhibitor is 2-(3,4-dihydroxyphenyl)-3,7-dihydroxy-4H-chromen-4-one (Fisetin) or salts thereof.

In certain embodiments, the subject is under 15 years, 10 years, or 5 years of age. In certain embodiments, the STAT3 and YB-1 inhibitors are administered before, during or, after radiation therapy.

In certain embodiments, the subject has received a first chemotherapy regiment and the brain cancer progresses despite the first chemotherapy regiment. In certain embodiments, the first chemotherapy regiment is a combination of cisplatin and 4-hydroxycyclophosphomide.

In certain embodiments, the STAT3 and YB-1 inhibitors are administered in combination with another chemotherapy agent. In certain embodiments, the chemotherapy agent is selected from abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, rib ociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, and zoledronic acid.

In certain embodiments, this disclosure contemplates ex vivo methods of diagnosing and treating medulloblastoma comprising a) culturing a sample of medulloblastoma on a culture media providing medulloblastoma; b) contacting the medulloblastoma with a chemotherapy agent or combination of chemotherapy agents; c) determining a quantity of cell death in the medulloblastoma with chemotherapy agent or combination of chemotherapy agents, and d) administering and effective amount of chemotherapy agent or combination chemotherapy agents to a subject in need thereof, wherein chemotherapy agent or combination chemotherapy agents are selected as having the largest quantity of cell death in the medulloblastoma.

In certain embodiments, this disclosure contemplates ex vivo methods of diagnosing and treating medulloblastoma comprising a) culturing a sample of medulloblastoma on a culture media providing medulloblastoma; b) contacting the medulloblastoma with a STAT3 inhibitor in combination with an YB-1 inhibitor; c) determining a quantity of cell death in the medulloblastoma with the combination of inhibitors compared to a control, and d) administering and effective amount of a STAT3 inhibitor in combination with an YB-1 inhibitor to a subject in need thereof, wherein the combination of inhibitors are selected as having the largest quantity of cell death in the medulloblastoma.

In certain embodiments, the culture media does not contain the amino acids glutamate and aspartate. In certain embodiments, the sample of medulloblastoma is from a human.

In certain embodiments, the STAT3 inhibitor is 3-(6-bromopyridin-2-yl)-2-cyano-N-(1-phenylethyl)acrylamide (WP1066) or salts thereof and YB-1 inhibitor is 2-(3,4-dihydroxyphenyl)-3,7-dihydroxy-4H-chromen-4-one (Fisetin) or salts thereof.

In certain embodiments, this disclosure relates to pharmaceutical compositions comprising a STAT3 inhibitor such as 3-(6-bromopyridin-2-yl)-2-cyano-N-(1-phenylethyl)acrylamide (WP1066) or salts thereof and YB-1 inhibitor is 2-(3,4-dihydroxyphenyl)-3,7-dihydroxy-4H-chromen-4-one (Fisetin) or salts thereof, and a pharmaceutically acceptable excipient.

In certain embodiments, the pharmaceutical composition is in the form of a pill, tablet, capsule, or gel. In certain embodiments, the pharmaceutical composition is in the form of a buffered isotonic saline solution. In certain embodiments, the pharmaceutically acceptable excipient is selected from stearic acid, magnesium stearate, calcium stearate, calcium phosphate, iron oxide, carnauba wax, sucrose, lactose, starch, sodium starch glycolate, cellulose, microcrystalline cellulose, methyl cellulose, ethyl cellulose, hydroxypropyl cellulose, hydroxypropyl methylcellulose, croscarmellose sodium, xylitol, sorbitol, mannitol, gelatin, sodium lauryl sulfate, polyvinylpyrrolidone, polyethylene glycol, silica, titanium dioxide, silicone dioxide, talc, magnesium carbonate, vitamin A, vitamin E, vitamin C, retinyl palmitate, and selenium, cysteine, methionine, citric acid, sodium citrate, methyl paraben and propyl paraben.

In certain embodiments, this disclosure relates to the production of a medicament for use in the treatment of brain cancer, e.g., medulloblastoma, in a human subject wherein the medicament comprises a STAT3 inhibitor such as 3-(6-bromopyridin-2-yl)-2-cyano-N-(1-phenylethyl)acrylamide (WP1066) or salts thereof and a YB-1 inhibitor such as 2-(3,4-dihydroxyphenyl)-3,7-dihydroxy-4H-chromen-4-one (Fisetin) or salts thereof, and a pharmaceutically acceptable excipient.

### BRIEF DESCRIPTION OF THE DRAWINGS

FIG. 1A shows data indicating activated STAT3 localizes to the perivascular niche in human and mouse medulloblastoma (MB). Immunostaining of patient-derived SHH MB showing positive phosphorylated (activated) STAT3 (pSTAT3) staining of tumor cells located adjacent to endothelial cells (Magnification 20×) (Arrows indicate the regions around blood vasculature)

FIG. 1B shows immunostaining of SmoA1 murine MB demonstrates co-localization of positive pSTAT3 and CD15 (tumor stem-like cell marker) adjacent to CD31 (endothelial marker). GFAP positive staining for normal astrocytes shows that normal astrocytes do not express activated STAT3 (Scale bar=50 μm). (Arrows indicate regions of co-localization)

FIG. 1C shows a schematic representation of the hypothesized interaction between STAT3 and YB-1 in the maintenance of MB cancer stem cell survival, and the respective inhibitors tested, WP1066 and fisetin.

FIG. 2A shows data indicating WP1066 treatment inhibits proliferation of SHH activated MB cells (DAOY, UW226, ONS-76), IC₅₀=1.8 μM, and a Group 3-like MB cell (0556), IC₅₀=1.5 μM. Treatment of human MB cells with WP 1066 and fisetin significantly decreases proliferation and inhibits colony formation.

FIG. 2B shows data indicating Fisetin treatment inhibits proliferation of SHH-activated MB cells, IC₅₀=3.8 μM, and 0556, IC₅₀=3.6 μM.

FIG. 2C shows data indicating WP1066 treatment inhibits colony formation of SHH-activated MB cells (composite results of DAOY, UW226. ONS-76) at concentration ≥1.5 μM (p<0.01) compared to vehicle control (DMSO).

FIG. 2D shows data indicating Fisetin treatment inhibits colony formation of SHH-activated MB cells at concentration ≥6 (p<0.01) compared to vehicle control (DMSO).

FIG. 2E shows data indicating cisplatin inhibits colony formation of SHH-activated MB cells at ≥2 μM compared to vehicle control (0.9% NaCl).

FIG. 2F shows data indicating cyclophosphamide derivative 4-hydroxycyclophosphamide (4HC) inhibits colony formation of SHH-activated MB cells at concentration ˜3 μM (p<0.01) compared to vehicle control (DMSO).

FIG. 3A illustrates the generation of the OSC tested: 1) Tumors are removed and placed in agarose gel, 2) Block of gel with tumor is placed on the base of a vibratome, 3) 300 μM slices are made and placed in a cell insert in a 6-well plate, with media beneath and the surface exposed to air. The slices are placed in an incubator for 24 hours, and then inhibitors/chemotherapeutics are added to the media and incubated for an additional 24 hours. The slices are then fixed, stained and analyzed using confocal microscopy.

FIG. 3B shows representative confocal images of SmoA1 OSC illustrating increasing amounts of cell death (represented by cleaved caspase 3 (CC3) staining) with increasing amount of WP1066 and fisetin.

FIG. 3C shows data on the quantification of CC3 staining observed in the OSCs showing significantly increased cell death following treatment with in WP1066 relative to DMSO vehicle Negative control treatment.

FIG. 3D shows data on fisetin. Treatment of SmoA1 murine SHH-activated MB organotypic slice culture (OSC) with WP1066 and fisetin induces marked tumor cell death.

FIG. 3E shows data for induction of tumor cell death compared to standard chemotherapy positive control treatment with either cisplatin.

FIG. 3F shows data with 4HC.

FIG. 4A shows data on the combined WP1066 and fisetin treatment significantly decreases proliferation of SHH activated human MB cells in vitro (composite results of DAOY, UW226, ONS-76).

FIG. 4B shows data on the combined treatment of SmoA1 OSCs: with WP1066 and fisetin significantly increases tumor cell death.

FIG. 4C shows in upper panel show representative immunostaining performed following treatment of SmoA1 OSCs with WP1066 and fisetin shows increased diffuse positive staining for cleaved caspase-3 (CC3) with combined treatment compared to single drug treatment alone. Lower panel: Quantification of CC3 and nestin positive co-staining (X-axis=intensity of nestin and Y-axis=intensity of CC3 positive staining; with the percentage of dead nestin-positive cells indicated in the upper right) demonstrates that fisetin and WP1066 combined treatment synergistically kills MB tumor stem-like cells, 25.36% CC3/fisetin-positive cells vs. 1.6% and 1.4% CC3/nestin-positive cells with either fisetin or WPW66 treatment alone respectively. DAPI=nuclear stain, DMSO=vehicle. Treatment of human MB cells and SmoA1 OSCs with WP1066 in combination with fisetin further enhances the inhibition of cell proliferation in vitro induction of tumor cell death diffusely ex vivo, but synergistically kills the tumor stem-like cell population specifically.

FIG. 5A shows representative images showing that increased cell death (CC3 positive staining) is similar following treatment with WPL066, fisetin, or cisplatin standard chemotherapy in comparison to DMSO vehicle negative control treatment.

FIG. 5B shows data on the quantification of immunostaining shown demonstrating an increased cell death in the treatment groups compared to the DMSO and sodium chloride vehicle negative controls. Treatment of a patient-derived non-WNT/SHH MB OSC with WPJ066 and fisetin induces tumor cell death

FIG. 5C is a schematic showing how preclinical drug screening for potential clinical efficacy is currently conducted vs. a proposed method using patient-derived OSC assay for rapid real-time evaluation of agents generating a drug activity readout within a clinically useful time to impact inhibitor/chemotherapy choice for the individual patient.

### DETAILED DESCRIPTION

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

The term “subject” refers to any animal, preferably a human patient, livestock, or domestic pet.

As used herein, the terms “treat” and “treating” are not limited to the case where the subject (e.g. patient) is cured and the disease is eradicated. Rather, the present disclosure also contemplates treatment that merely reduces symptoms, and/or delays disease progression.

As used herein “cancer” refers any of various cellular diseases with malignant neoplasms characterized by the proliferation of cells. Most cancers form a tumor but some, like leukemia, do not. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed. It may also be identified by a change in relevant biomarker or gene expression profile, such as HER2 for breast cancer.

Medulloblastoma is a type of malignant brain tumor (cancer) typically identified in the part of the brain that is towards the back and the bottom or the skull, in the small cerebellum. Medulloblastomas are capable of spreading through the cerebrospinal fluid and metastasizing to different locations along the surface of the brain and spinal cord. Medulloblastoma are typically distinguished from other CNS embryonal tumors and embryonal tumors that display multilayered rosettes, e.g., LIN28A is typically expressed, sometimes harbor an amplification on chromosome 19 of a large miRNA cluster (C19MC), and atypical teratoid rhabdoid tumors, which are typically indicated by a loss of SMARCB1 (INI1) loss or of SMARCA4 (BRG1).

Medulloblastoma may be characterized by four distinct molecular and clinical variants which are typically identified by gene mutations in variant biological pathways termed WNT/β-catenin, Sonic Hedgehog (SHH), Group 3, and Group 4 (WNT, SHH, and WNT/SHH-independent medulloblastoma disease subgroups, GABAergic and glutamatergic gene expression). WNT histology is typically classic and rarely large cell, and may be indicated by of somatic mutations of CTNNB1 encoding b-catenin. SHH (Sonic hedgehog) histology is typically desmoplastic/nodular, classic, large cell, and may be indicated by SHH receptor PTCH mutations, SHH inhibitor SUFU mutations, and/or amplifications of GLI1 and GLI2. Group 3 histology is typically classic, large cell, and may be indicated by MYC amplification. Group 4 histology is typically classic, large cell, and may be indicated CDK6 and MYCN amplification. In certain embodiments, this disclosure contemplates that in any of the treatment methods disclosed herein, the subject might be diagnosed with one of the medulloblastoma subtypes.

As used herein a “culture medium” or “media” refers to a composition that contains components, such as vitamins, amino acids, inorganic salts, a buffer, and a fuel, e.g., acetate, succinate, and/or a saccharide, that support the growth and maintenance of cell lines. Components in the culture medium may be derived from blood serum or may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Brewer et al. report, in the Journal of Neuroscience Research 35:567-576 (1993), a commercial product with concentrations of each component optimized for neuron survival. The medium contains vitamins, essential fatty acids, hormones, and antioxidants. Described are benefits of a lower glutamine concentration, reduced osmolarity, and toxicity of ferrous sulfate. Further considerations to avoid excitatory amino acid toxicity containing no glutamate or aspartate. Thus, in certain embodiments, this disclosure contemplates that neuronal culture media with less than 1 mg/L of iron salts, glutamate, or aspartate.

An Ex Vivo Model for Medulloblastoma Therapy Development Demonstrates that Targeting STAT3 and YB-1 Synergizes to Drive Cell Death in the Tumor Stem-Like Cell Population

Medulloblastoma (MB) is the most common malignant pediatric brain tumor. Modem treatments result in cognitive and physical disabilities; thus, more effective tumor-targeting strategies are needed. Based on their expression in the radiation resistant tumor stem cell population in the Sonic hedgehog (SHH) subclass of medulloblastoma, targeting STAT3 and YB-I using two inhibitors was tested and compared to standard chemotherapy (cisplatin and 4-hydroxycyclophosphamide (4HC)) in an organotypic ex vivo brain tumor slice culture (OSC) to determine the interaction between STAT3 and YB-1 in MB, establish the pre-clinical efficacy of WP1066 and fisetin, and validate our OSC assay. WP1066 and fisetin treatment of human sonic hedgehog (SHH) MB cells inhibited proliferation (IC₅₀ of 1.8 μM and 3.8 μM) and colony formation assay (CPA) in a dose dependent manner. In murine SHH MB OSC assays, a dose-dependent increase in cell death was observed with WP1066 and fisetin similar to 4HC or cisplatin treatment. Combined WP1066 and Fisetin treatment had an additive effect overall, but was synergistic in OSC for killing tumor stem-like cells in the perivascular niche (PVN), where therapy resistant tumor re-populating cells reside. WP1066 and Fisetin treatment also induced cell death in an OSC assay of a patient-derived non-SHH/non-WNT MB obtained at diagnosis. The in vitro results correspond directly to our ex vivo model, and OSC assay was performed using a patient-derived MB, illustrating the feasibility of our OSC assay for patient tailored therapy, as well the therapeutic potential for targeting YB-1 and STAT3 in medulloblastoma.

Drug testing using patient-derived medulloblastoma OSC will be a reliable assay for the identification of effective therapies tailored to each molecular subgroup, as well as a screening tool to aid in the selection of agents tailored to the individual patient. Two molecularly targeted drug treatments were evaluated for future clinical investigation against SHH MB, WP 1066 [Signal transducer and activator of transcription 3 (STAT3) inhibitor) and fisetin [Y-box 1 (YB-I) inhibitor]. These treatments were compared to standard chemotherapy for medulloblastoma using classical preclinical drug testing assays (colony forming assay (CFA) and proliferation assay) to first establish the relative in vitro anti-tumor efficacy of these drugs, and then tested the same drugs in an OSC assay derived from the SmoA1 transgenic mouse model of SHH-activated medulloblastoma and a human Group 3 specimen to confirm efficacy of these novel treatments in an intact living 3-dimensional tumor environment.

These targets were chosen because (1) YB-1 is up-regulated across all MB molecular subclasses; (2) YB-1 localizes to the PVN in human SHH 1\4B; (3) STAT3 is an oncogene implicated in the maintenance of cancer stem-like cells and has been reported to be both highly expressed and activated in 1\4B. STAT3 was found to be highly expressed in the cancer stem-like cell population of murine SHH MB, and inhibition of STAT3 has been shown to induce MB cell apoptosis and promote treatment resistance and tumor recurrence. WP1066 is a small molecule inhibitor of the JAK/STAT3 pathway. YB-1 is part of the Yes Associated protein (YAP): YB-1: Insulin-like Growth Factor 2 (IGF2) signaling axis. YB-1 is a pleiotropic DNA and RNA-binding protein. YB-1 drives proliferation in SHH-dependent MB cells and is required for expression of the survival promoting signaling molecule Insulin-like Growth Factor 2 (IGF2). Fisetin (3,7,3′,4′-tetrahydroxyflavone) is a bioactive flavonoid that inhibits cell survival in numerous cancers and inhibits YB-1 through binding to its site of phosphorylation and blocking Akt-mediated phosphorylation at Serine 102, which is required for YB-1's nuclear functions. A preferred embodiment of this disclosure is the use of WPI066 and fisetin as agents, alone and in combination, for the treatment of medulloblastoma.

Treatment with the STAT3 inhibitor WP1066 and the YB-I inhibitor fisetin significantly inhibits colony formation, and induces cell death in the OSC assay. The effects on the tumor by each drug alone is similar to, and may prove to be better than, cisplatin and cyclophosphamide, currently used in treatment regimens for MB. Combining WP1066 and fisetin enhances tumor cell death, and this effect is synergistic specifically against the tumor stem-like cell population, which is thought to be responsible for treatment-resistance, metastasis and tumor recurrence.

### Examples

### Materials

Daoy, UW228, ONS-76, and 0556 human MB cells were obtained from the ATCC (Manassas, Va.). Daoy and UW228 cells are SHH-activated and TP53 mutated. ONS-76 is SHH-activated and TP53 wild-type. D556 cells are MYC amplified and were obtained from a patient with anaplastic MB, and therefore is most similar to Group 3 MB. WP1066 stock solution 2 mM was made by diluting the powdered drug in dimethyl sulfoxide (DMSO) and storing at −20 C. Fisetin stock 1 mM was made by diluting the powdered drug in DMSO and stored at −20 C. Cisplatin stock solution 1 mM was made by diluting the powdered drug in normal saline and was used fresh. 4-Hydroxycyclop11ospbomide (4HC) stock solution 1 mM was made by dissolving in DMSO and stored at −20 C.

### Ex Vivo Brain Tumor Slice Culture

For the ex vivo experiments, SmoA1 and Math-Cre-ER-Ptch^{f/f} mice were used. The SmoA1 mice spontaneously develop tumors and were used between 4-6 months of age. For the Math-Cre-ER-Ptch^{f/f} model, pregnant mice were administered Tamoxifen (20 mg Tamoxifen per 1 mL com oil, dose is 4 mg oral gavage×1 dose) after E17.0 to induce MB formation. Tumor-bearing mice were sacrificed using CO₂ euthanasia when symptomatic with tumor.

For human medulloblastoma, samples were taken directly from the operating room at time of initial resection. Tumors were sectioned horizontally into 300 μm thick slices with a vibratome. The slices were cultured on trans-well inserts (1 μm pore size) in Neurobasal® medium supplemented with B27. Neurobasal®, when supplemented with B-27® Supplement, contain antioxidants to reduce reactive oxygen damage and does not contain the excitatory amino acids, glutamate and aspartate.

After 24 hours in culture, increasing concentration of WP 1066, fisetin, cisplatin, and 4HC were added to the media, and then the slices were cultured for 24 hrs. After 48 hrs total hours, the slices were fixed in 4% paraformaldehyde and immunostained with antibodies against cleaved caspase 3. Secondary antibodies and DAPI were applied and confocal images were obtained using a laser confocal microscope and captured for analysis and quantification.

### High Expression of Total and Active STAT3 and YB-1 Co-Localizes in Human and Murine SHH MB.

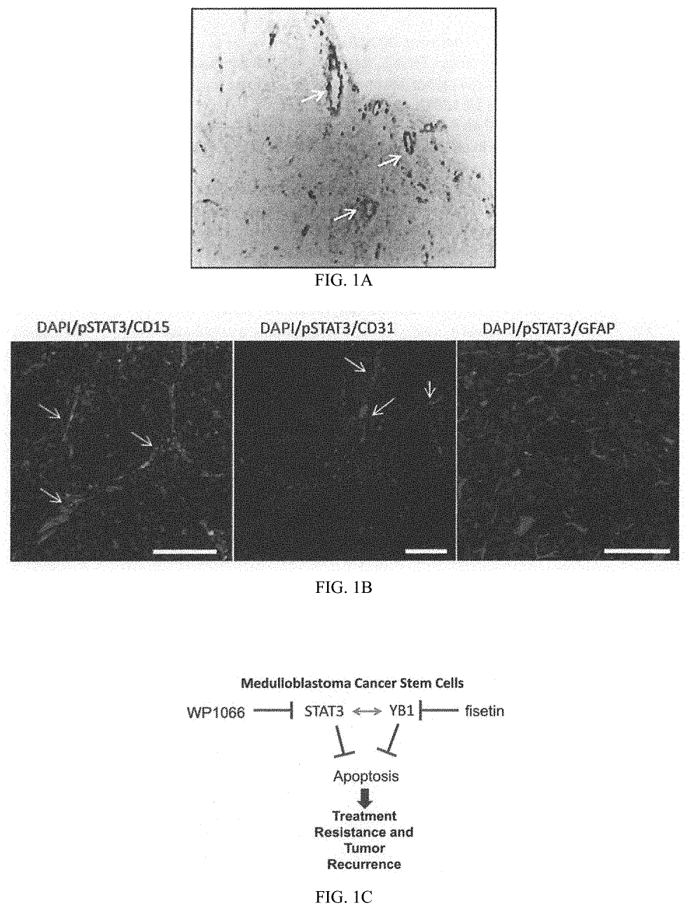

YB-I expression is upregulated across all MB subgroups and YB-1 is present at high levels in the PVN of human SHH MB. Experiments were performed to determine whether STAT3 and phospho-STAT3 (pSTAT3) localizes to the perivascular niche (PVN) in human and murine SHH driven MB. On a tissue microarray human MB specimens were generated. Routine IHC for pSTAT3 was evaluated on 32 specimens. Thirty of thirty-two (30/32)(94%) specimens were positively stained across subgroups (WNT, SHH, Group 3 and Group 4). The highest level of expression of total and p-STAT3 localized to the PVN (FIG. 1A). Immunostaining of SmoA1 was performed on a MB cut section for the MB tumor stem-like cell marker CD15, endothelial marker CD31, and pSTAT3, confirming expression of pSTAT3 in the tumor stem-like cells of the perivascular niche (FIG. 1B). Normal GF AP positive astrocytes did not stain positive for pSTAT3 indicating that STAT3 activation is restricted to the tumor cells. Thus, a contemplated model is the interaction between YB-1 and STAT3 for the maintenance of the MB cancer stem cell, which promotes resistance to therapy and tumor recurrence (FIG. 1C).

### MB Cell Proliferation and Colony Formation is Inhibited by WP1066 and Fisetin Treatment.

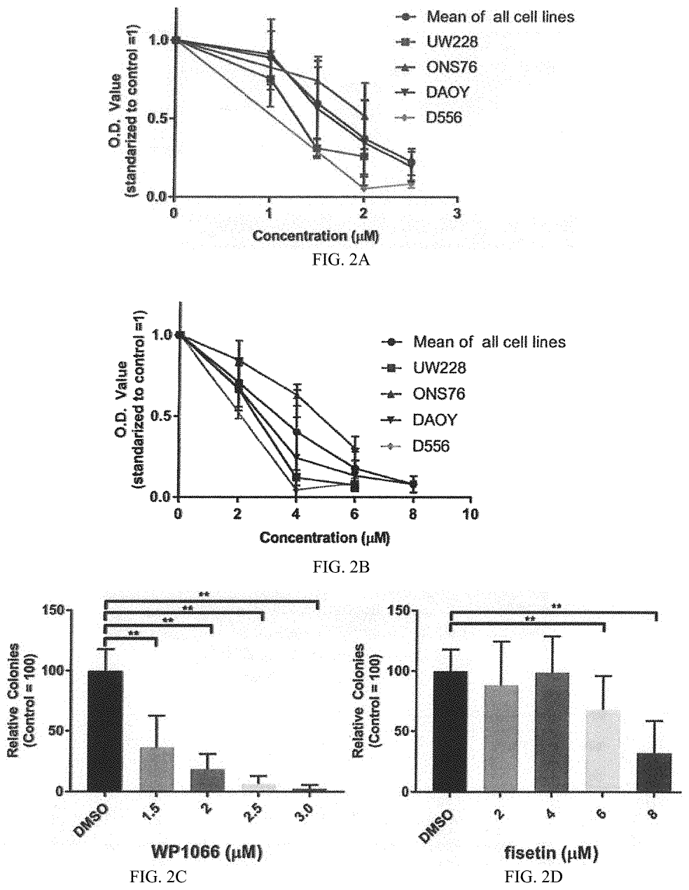

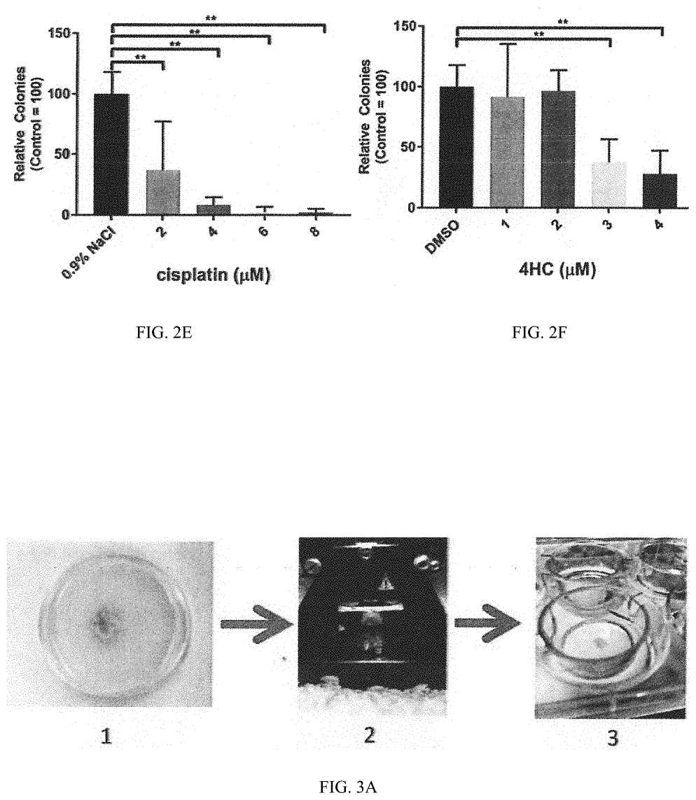

The effect of STAT3/YB-1 inhibition on human MB cell lines was investigated. WP1066 or fisetin treatment inhibited proliferation of SHH activated MB cells (DAOY, UW228 and ONS-76) in a dose-dependent manner, with an IC₅₀ of the mean of all SHH activated cells to be 1.8 μM and 3.8 μM, respectively (FIG. 2A, B). To evaluate the effect on non-SHH activated MB cells, inhibition of proliferation of the Group 3-like D556 MB cells was tested, and an IC₅₀ concentration of 1.2 μM for WP1066 and 3.6 μM for fisetin was observed (FIG. 2A, B). The effect of increasing drug treatment concentrations using on the stem-like tumor cell population and tumor initiation in vitro was evaluated using a soft agar colony formation assay (CFA). WP1066 and fisetin inhibited colony formation of DAOY, UW228 and ONS-76 in a dose-dependent manner when compared to control (FIG. 2C, D). In comparison to standard chemotherapy, for MB (cisplatin, 4HC), WP1066 or fisetin alone demonstrated similar inhibition of colony formation (FIG. 2E, F).

### WP1066 and Fisetin Induce Marked Cell Death in Ex Vivo Organotypic Slice Cultures

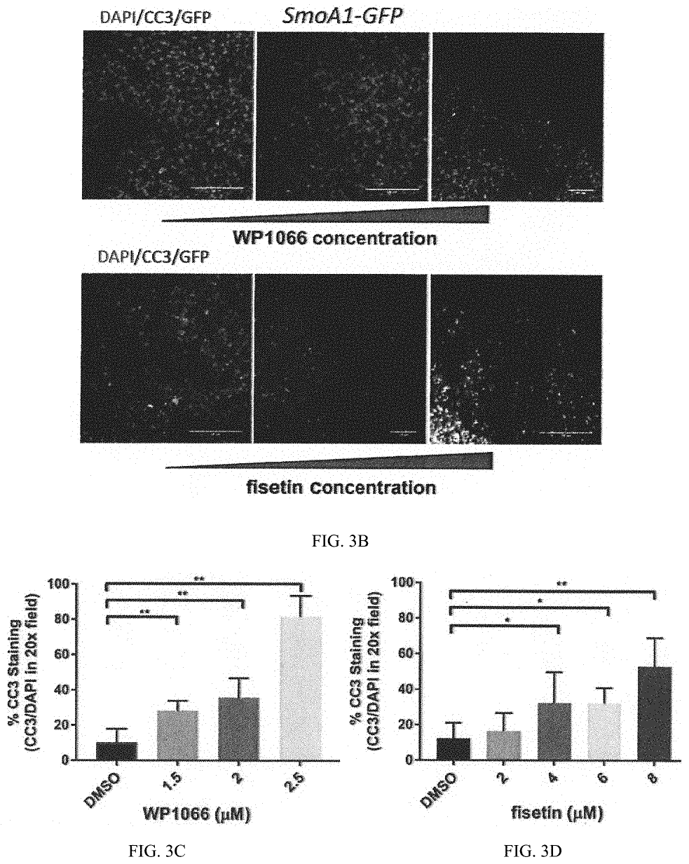

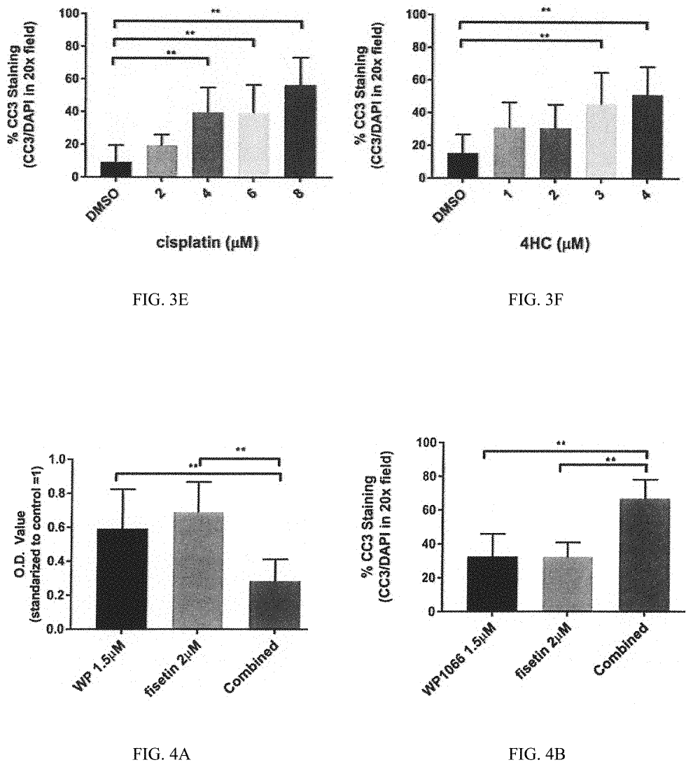

WP1066, fisetin, cisplatin, and 4HC were evaluated in murine SmoAI and Math-Cre-ER-Ptch^{f/f} MB tumor slice cultures. OSCs were grown for 24 hours and then treated with escalating, doses of WP1066 and fisetin (FIG. 3A). After an additional 24 hours, the cells were fixed and immunostained with cleaved caspase 3 (CC3) as a marker of cell death. After quantifying, the amount of CC3 standardized to the amount of DAP1 positive staining, a significant dose-dependent increase in cell death was found with either WP1066 or fisetin concentrations greater than 1.5 μM and 4 μM, respectively when compared to control (FIG. 3B, C). To further validate the assay as a reliable preclinical drug screening tool, the results obtained were compared with WP1066 and fisetin to standard chemotherapy treatments with in vivo activity against medulloblastoma: 4HC, or cisplatin. Treatment with 4HC and cisplatin led to significant cell death for each at a concertation of 2 μM similar to the effects of the inhibitors tested relative to treatment with vehicle control alone. Chemotherapies with minimal in vivo activity were tested against MB (Busulfan and 6-thioguinine) in the ex vivo OSC system. Corresponding minimal increase in cell death relative to vehicle controls were found. These results indicate that the OSC model closely recapitulates known in vivo experiments for historic drugs with known effects, while the inhibitors WP1066 and fisetin show similar efficacy.

### WP1066 and Fisetin Synergize to Drive Tumor Stem-Like Cell Death

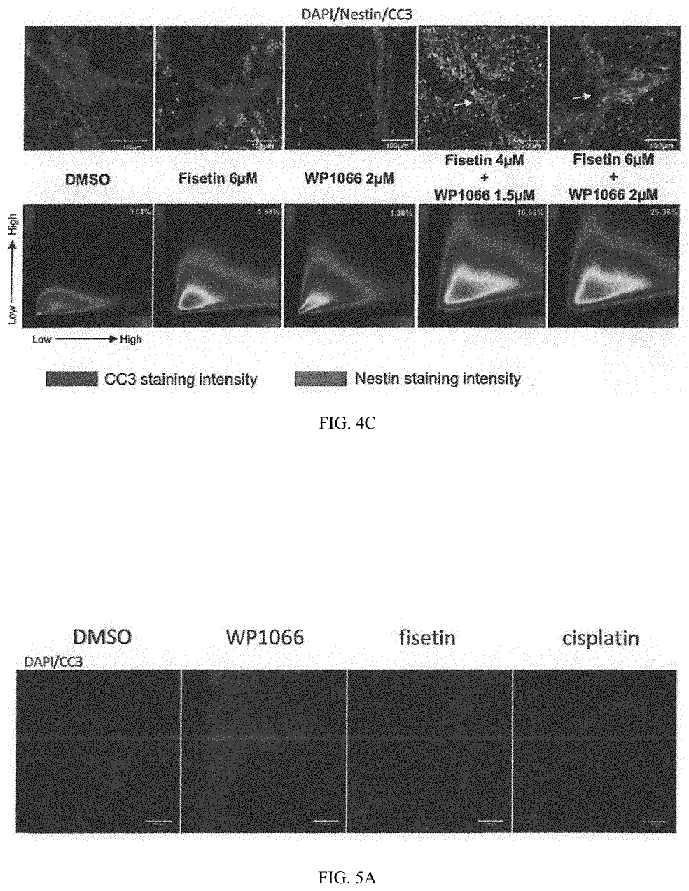

Given that STA 3 and YB-1 expression colocalized to the cancer stem cell-like cells of the perivascular niche environment, experiments were performed to determine whether combined treatment with these inhibitors, fisetin and WPHH66, leads: to increased tumor cell and/or tumor stem cell like cell death. The effect of combination treatment was evaluated in the in vitro proliferation and colony formation assays. An additive effect was found on the inhibition of proliferation and colony formation in SHH-MB cell (FIG. 4A, B). Next, experiments were performed to determine whether this effect was more pronounced in the stem cell compartment, where YB-1 and STAT3 are most highly expressed. SmoA1 MB ex vivo brain slices were treated with WP1066, fisetin, or combined treatment thereof. The slices were immunostained for the tumor stem-like cell marker nestin and the cell death marker CC3 to quantify the amount of cell death in the tumor stem-like cells. Combined treatment with WP1066 and fisetin of ex vivo MB resulted in marked synergy in cell death of the tumor stem-like cells in the perivascular niche (FIG. 4C).

### Ex Vivo Tumor Slice Culture of Patient-Derived Non-WNT/SHH Medulloblastoma Responds to WP1066 and Fisetin Treatment

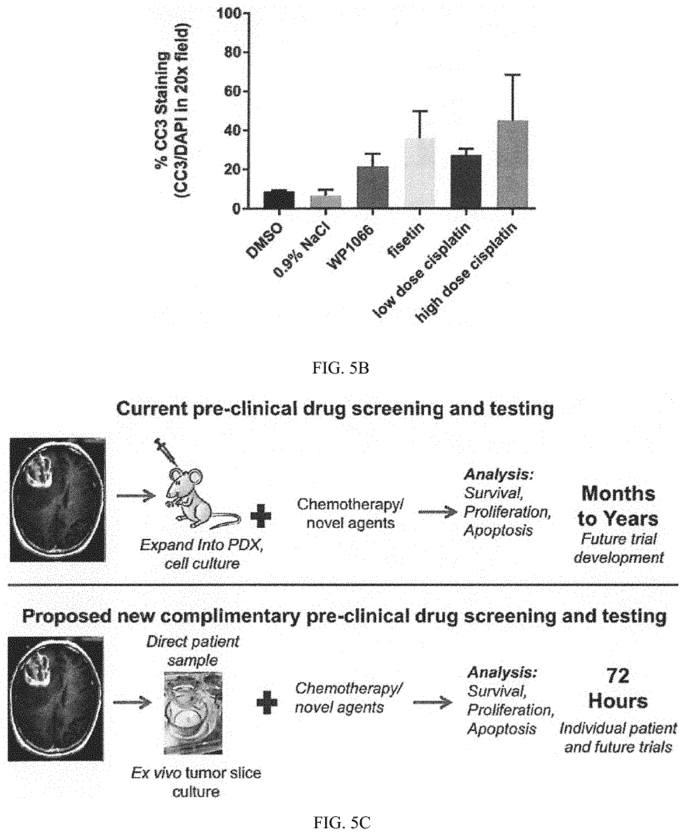

Ex vivo, cultures can be used to evaluate drug effects on murine MB tumor cells in their microenvironment. Experiments were performed to determine whether OSC could be used as a “precision medicine” technique in medulloblastoma, e.g., whether biopsy sample from either a newly diagnosed or relapsed medulloblastoma patient could be directly obtained in the operating room, transferred to the lab, and OSCs generated and tested per the protocol, with readout of the efficacy of potential treatments within 3 days. After obtaining fresh tumor tissue from a newly diagnosed patient with a cerebellar tumor, an OSC assay using this patient-derived medulloblastoma specimen was performed. Four tumor slices were treated with vehicle (DMSO or PBS) and 6 tumor slices were treated with WP1066, fisetin, and cisplatin as positive control. Increase in cell death with treatment of WP1066, fisetin, or cisplatin were observed relative to vehicle controls (FIG. 5 A, B). Therefore this assay can be used at the time of diagnosis, indicating the feasibility of OSC for use in a personalized medicine based testing approach (FIG. 5C) in pediatric brain tumors. Later the neuropathology report confirmed isochromosome 17q in the specimen without MYC or MYCN amplification consistent with a non-WNT/SHH MB.

1. A method of treating medulloblastoma comprising administering an effective amount of a STAT3 inhibitor in combination with an YB-1 inhibitor to a subject in need thereof.

2. The method of claim 1, wherein the subject is diagnosed with medulloblastoma.

3. The method of claim 1, wherein the STAT3 inhibitor is 3-(6-bromopyridin-2-yl)-2-cyano-N-(1-phenylethyl)acrylamide (WP1066) or salt thereof and YB-1 inhibitor is 2-(3,4-dihydroxyphenyl)-3,7-dihydroxy-4H-chromen-4-one (Fisetin) or salt thereof.

4. The method of claim 1, wherein the subject is a human.

5. The method of claim 4, wherein the subject is under 15 years, 10 years, or 5 years of age.

6. The method of claim 1, wherein the STAT3 and YB-1 inhibitors are administered before, during or, after radiation therapy.

7. The method of claim 1, wherein the subject has received a first chemotherapy regiment and the brain cancer progresses despite the first chemotherapy regiment.

8. The method of claim 7, wherein the first chemotherapy regiment is a cisplatin and 4-hydroxycyclophosphomide.

9. The method of claim 1, wherein the STAT3 and YB-1 inhibitors are administered in combination with another chemotherapy agent.

10. The method of claim 9, wherein another chemotherapy agent is selected from abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, rib ociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, and zoledronic acid.

11. A method of diagnosing and treating medulloblastoma comprising
a) culturing a sample of medulloblastoma on a culture media providing medulloblastoma;
b) contacting the medulloblastoma with a chemotherapy agent or combination of chemotherapy agents;
c) determining a quantity of cell death in the medulloblastoma with the chemotherapy agent or combination of chemotherapy agents, and
d) administering and effective amount of a chemotherapy agent or combination of chemotherapy agents to a subject in need thereof, wherein the chemotherapy agent or combination of chemotherapy agents are selected as having the largest quantity of cell death in the medulloblastoma.

12. The method of claim 11, wherein the culture media does not contain the amino acids glutamate and aspartate.

13. The method of claim 11, wherein the combination of chemotherapy agents are a STAT3 inhibitor or salt thereof in combination with an YB-1 inhibitor or salt thereof.

14. The method of claim 13, wherein the STAT3 inhibitor is 3-(6-bromopyridin-2-yl)-2-cyano-N-(1-phenylethyl)acrylamide (WP1066) or salt thereof and YB-1 inhibitor is 2-(3,4-dihydroxyphenyl)-3,7-dihydroxy-4H-chromen-4-one (Fisetin) or salt thereof.

15. The method of claim 11, wherein the sample is from a human subject.
